(19) United States
(12) Patent Application Publication
(10) Pub. No.: US 2009/0259171 A1
(43) Pub. Date: Oct. 15, 2009

(54) Transdermal Oxygen-Delivery Apparatus and Method

(76) Inventors: Ashok V. Joshi, Salt Lake City, UT (US); John Howard Gordon, Salt Lake City, UT (US)

Correspondence Address: CERAMATEC, INC., 2425 SOUTH 900 WEST, SALT LAKE CITY, UT, 84119, US

(21) Appl. No.: 12/421,464

(22) Filed: Apr. 9, 2009

Related U.S. Application Data
(60) Provisional application No. 61/043,689, filed on Apr. 9, 2008.
(60) Provisional application No. 61/078,225, filed on Jul. 3, 2008.

(51) Int. Cl. A61M 37/00

(52) U.S. Cl. 604/24; 604/23

(57) ABSTRACT

An apparatus and method for facilitating transdermal oxygen delivery is disclosed in one embodiment of the invention as including a supply source coupled to a delivery device. The supply source may provide a supply of oxygen that may be delivered transdermally through the skin of a patient via the delivery device. In selected embodiments, the delivery device may include a barrier layer to substantially retain the oxygen over a localized area of skin, and a gas-permeable contact layer to deliver the oxygen to the localized area. Finally, a transport enhancement element may increase the oxygen permeability of the localized area.

### RELATED APPLICATIONS

This application claims priority to U.S. Provisional Patent No. 61/043,689 filed on Apr. 9, 2008 and entitled DEVICE AND METHOD FOR RAISING OXYGEN TENSION IN SUBCUTANEOUS TISSUE, and to U.S. Provisional Patent No. 61/078,225 filed on Jul. 3, 2008 and entitled DEVICE TO PROVIDE EMERGENCY ARTIFICIAL RESPIRATION WITHOUT AN AIRWAY.

### FIELD OF THE INVENTION

This invention relates to apparatus and methods for delivering oxygen to a patient transdermally to increase subcutaneous oxygen tension.

### BACKGROUND

Adequate oxygen consumption is essential to maintaining human life and consciousness. Human lungs are uniquely equipped to facilitate the process of gas exchange in which oxygen, carbon dioxide, and other gases passively diffuse between alveoli in the lungs and blood passing by in the lung capillaries. Once in the blood, the heart powers the flow of oxygen and other dissolved gases through the body via the circulatory system.

Under normal conditions, humans can store very little oxygen in the body. Prolonged apnea leads to a severe lack of oxygen circulating throughout the body. This may result in permanent brain damage in as little as three minutes, after which death inevitably ensues unless the flow of oxygen is restored.

This prognosis is especially disturbing for affected individuals without access to immediate medical intervention. Indeed, the usual medical protocol for quickly restoring respiration in a person suffering from a damaged or collapsed trachea is to perform tracheal intubation. This procedure requires a trained professional to insert a tube through the trachea using a laryngeal scope, thereby artificially introducing oxygen into the lungs and facilitating a flow of carbon dioxide out of the lungs. Where the damage to the trachea arises from an allergic reaction or from an injury to the throat or neck on the battlefield, for example, medical personnel having the necessary training may not be immediately available. Further, the procedure may not be feasible due to the immediate danger the victim may be facing, and may be precluded by a lack of necessary equipment.

In addition to supporting life processes, oxygen also plays a vital role in wound healing. Specifically, oxygen is necessary for cell proliferation and angiogenesis, or the physiological process of growing new blood vessels from pre-existing vessels. Hypoxia, or an insufficient supply of oxygen, prevents normal healing processes.

Implanted cells or tissues are particularly prone to hypoxia due to insufficient or non-existent vascularization. For example, pancreatic islet cells transplanted from one animal to another for the purpose of controlling insulin levels may lack direct access to a blood supply. As a result, such cells may rely on the oxygen in surrounding plasma for metabolic requirements.

Direct application of oxygen to a wound resulting from trauma, surgery, burns, skin grafts, or cellular or tissue implantation may impart a variety of benefits. Such benefits may include eliminating hypoxia, reducing clinical infection and edema, and favorably influencing cytokine down regulation and growth factor up regulation.

Hyperbaric oxygen therapy involves exposing a subject to elevated pressures while breathing 100% oxygen and is often hailed as a means to increase wound healing. This treatment, however, has several disadvantages. For example, such treatment may cause ear and sinus barotraumas, myopia, aggravation of congestive heart failure, oxygen seizures, and pulmonary barotraumas. Additionally, subjects who have an untreated pneumothorax, severe obstructive pulmonary disease, untreated asthma, chronic obstructive pulmonary disease, or congestive heart failure may not be eligible for hyperbaric oxygen therapy treatment.

Moreover, the equipment needed to perform hyperbaric oxygen therapy is expensive, not portable, and requires an attendant to monitor therapy. Typical wound treatment requires numerous sessions, with associated expense and inconvenience as the person undergoing treatment must be transported to a clinic for each treatment session.

In view of the foregoing, what are needed are apparatus and methods to provide an immediate supply of oxygen locally or systemically without requiring tracheal intubation. Further needed are apparatus and methods to increase transdermal oxygen absorption and facilitate the removal of carbon dioxide from the body. Further needed are portable oxygen-generation and delivery devices to provide increased oxygen to damaged and implanted tissues and cells.

### SUMMARY OF THE INVENTION

The invention has been developed in response to the present state of the art and, in particular, in response to the problems and needs in the art that have not yet been fully solved by currently available oxygen delivery devices. Accordingly, the invention has been developed to provide novel apparatus and methods for delivering oxygen transdermally to promote respiration, angiogenesis and wound healing. The features and advantages of the invention will become more fully apparent from the following description and appended claims and their equivalents, and also any subsequent claims or amendments presented, or may be learned by practice of the invention as set forth hereinafter.

Consistent with the foregoing, an apparatus for facilitating transdermal oxygen delivery is disclosed in one embodiment of the invention as including a supply source coupled to a delivery device. The supply source may provide a supply of oxygen that may be delivered transdermally through the skin of a patient via the delivery device. In selected embodiments, the delivery device may include a barrier layer to substantially retain the oxygen over a localized area of skin, and a gas-permeable contact layer to deliver the oxygen to the localized area. Finally, a transport enhancement element may increase the oxygen permeability of the localized area.

A corresponding method is also disclosed and claimed herein.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 is a high-level block diagram of one embodiment of an apparatus for generating a supply of oxygen and delivering the oxygen through a localized area of skin;

FIG. 2 is a high-level block diagram of another embodiment of an apparatus for generating and delivering oxygen through the skin;

FIG. 3 is a high-level block diagram of an embodiment of an apparatus for providing a supply of oxygen and delivering the oxygen through the skin;

FIG. 4 is a high-level block diagram of one embodiment of a patch for transdermal oxygen delivery that includes an array of microneedles to increase skin permeabiltiy;

FIG. 5 is a high-level block diagram of one embodiment of a tool for perforating the skin to increase gas permeability;

FIG. 6 is a high-level block diagram of another embodiment of a patch for transdermal oxygen delivery that may be used in conjunction with the tool of FIG. 5;

FIG. 7 is a high-level block diagram of an embodiment of a patch for transdermal oxygen delivery incorporating a heat element to selectively increase skin temperature to enhance permeability;

FIG. 8 is a high-level block diagram of an alternative embodiment of the patch of FIG. 7;

FIG. 9 is a high-level block diagram of another embodiment of a patch for transdermal oxygen delivery; and

FIG. 10 is a flow chart detailing steps for facilitating transdermal oxygen delivery in accordance with embodiments of the invention.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

For the purposes of this description, the term “stratum corneum,” as used herein, refers to the topmost layer of mammalian skin. The term “transdermal” refers to the absorption or application of oxygen into regions or tissues residing beneath the stratum corneum, including into the bloodstream. The term “subcutaneous” or “subdermal” refers to regions or tissues residing beneath the stratum corneum.

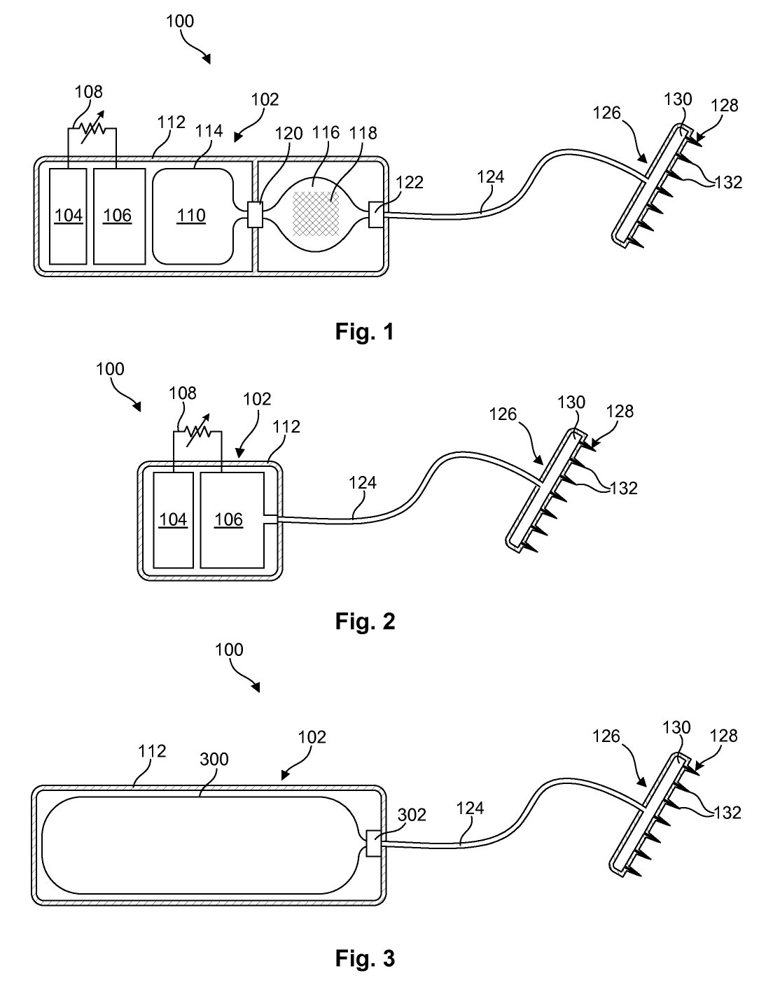

Referring now to FIG. 1, an apparatus 100 for facilitating transdermal oxygen delivery in accordance with embodiments of the invention may include a supply source 102 to provide a supply of oxygen and a delivery device 126 to deliver the oxygen transdermally through the skin of a patient. A transport enhancement element 128 may increase skin permeability, thereby increasing transdermal oxygen transport. Such an apparatus 100 may thus promote wound healing and/or sustain life, depending on the quantity and rate at which oxygen is transdermally supplied and distributed. The apparatus 100 may be in the form of a bandage.

In some embodiments, the supply source 102 may include a substantially rigid housing 112 containing a battery 104 coupled to a gas-generating cell 106. Upon initiating an electrical switch (not shown), the battery 104 may provide electrical current to the gas-generating cell 106. In some embodiments, a gas-generator circuit 108 communicating with the battery 104 and gas-generating cell 106 may include an adjustable resistor to enable selective variation and control of the electrical current flowing from the battery 104 to the gas-generating cell 106.

A flexible enclosure 110 may be situated within the housing 112 proximate the gas-generating cell 106, and may contain a solution such as a stabilized 30% aqueous hydrogen peroxide solution. In other embodiments, the flexible enclosure 110 may contain any solution known to those in the art able to produce oxygen upon reacting with a catalyst.

In operation, the gas generated by the gas-generating cell 106 may be retained within the substantially rigid housing 112. As the volume of the gas within the housing 112 increases, the flexible walls 114 of the enclosure 110 may become compressed. This compression may force the solution to flow from the enclosure 110 into a reaction chamber 116 coupled thereto. A check valve 120 may prevent back flow from the reaction chamber 116 to the enclosure 110.

The reaction chamber 116 may contain a catalyst 118, such as silver mesh. Upon reaching the reaction chamber 116, the solution may react with the catalyst 118 to generate oxygen. In one embodiment, a hydrogen peroxide solution may contact a silver mesh catalyst 118 to generate oxygen according to the following decomposition reaction:

H₂O₂→H₂O+½ O₂

In other embodiments, other chemical reactions known to those in the art may be utilized to generate oxygen.

The reaction chamber 116 may contain a sufficient amount of the catalyst 118 to ensure that the oxygen generation rate is dependent only on the amount of solution entering the chamber 116, rather than the decomposition rate. In some embodiments, the reaction chamber 116 may include substantially flexible or elastic sidewalls to enable the volume of the reaction chamber 116 to expand to accommodate water and/or other byproducts of the oxygen-generating reaction.

Oxygen gas produced by the reaction may proceed through a filter 122 attached to the reaction chamber 116. The filter 122 may include, for example, a microporous fluorinated polymer to contain water droplets within the reaction chamber 116 while enabling oxygen gas to pass through. Alternatively, the filter 122 may include any other suitable material known to those in the art.

The oxygen gas may proceed through the filter 122 to the delivery device 126. In one embodiment, the delivery device 126 may include a substantially flexible oxygen-supply line 124 coupled to a delivery chamber 130. A transport enhancement element 128, such as an array 132 of hollow microneedles, may be coupled to the delivery device 126 to facilitate oxygen permeation and diffusion through the skin upon delivery.

In certain embodiments, the array 132 may include microneedles having dimensions ranging between about ten to about one thousand microns in length, with cross-sectional dimensions ranging between about ten and about one hundred microns. Hollow microneedles may include inner diameters ranging between about three and about eighty microns. The microneedles may be fabricated in the array 132 and connected to a flexible sheet. Further, in certain embodiments, the microneedles may be fabricated with wider bases and narrow tips to penetrate skin easily and substantially painlessly without breaking. In some embodiments, the microneedles may be fabricated from biopolymers that decompose in the body. In this manner, the microneedles may be eventually absorbed into the body if they happen to break off while inserted.

Specifically, in certain embodiments, the apparatus 100 may be activated with a switch (not shown) and the microneedle array 132 may be pushed onto exposed skin on the body surface. The rate of oxygen generation may be determined by the voltage of the batteries and the resistance in the gas-generator circuit 108. Upon exiting the filter 122 as previously discussed, the oxygen gas may pass through the array 132 of microneedles to enter subcutaneous tissues and regions at a rate substantially determined by the gas-generating cell 106. The oxygen may be absorbed by fluids in the body and, in some embodiments, may diffuse under a concentration gradient into the circulatory system. The oxygen may then be distributed throughout the body via circulation.

Referring now to FIG. 2, an alternative embodiment of the supply source 102 in accordance with the present invention may include a battery 104 coupled to a gas-generating cell 106 via a gas-generator circuit 108. The battery 104, gas-generating cell 106, and gas-generator circuit 108 may reside within a housing 112. As above, in certain embodiments, the amount of gas generated by the gas-generating cell 106 may be regulated by an adjustable resistor or other current-regulating device.

In the embodiment shown in FIG. 2, the gas-generating cell 106 may include an electrochemical cell configured to directly produce oxygen gas. In one embodiment, for example, the electrochemical cell may include a solid oxide electrolyte membrane. The oxygen gas produced may flow directly into the delivery device 126 for transdermal delivery.

As shown, the delivery device 126 may include an oxygen-supply line 124 and a delivery chamber 130. The oxygen-supply line 124 may direct the oxygen gas from the gas-generating cell 106 to the delivery chamber 130. The delivery chamber 130 may temporarily retain the oxygen gas prior to transdermal delivery.

In some embodiments, a transport enhancement element 128 may be attached to the delivery chamber 130 to facilitate transdermal oxygen delivery. In one embodiment, an array 132 of substantially hollow microneedles may be attached to the delivery chamber 130 such that the oxygen gas may be received into the array 132 and exit subcutaneously through the hollow microneedles.

In operation, the array 132 of microneedles may be pressed against an area of skin to increase skin permeability and facilitate subdermal oxygen reception by enabling a flow of oxygen to effectively bypass the stratum corneum. The battery 104 and gas-generating cell 106 may be actuated to instigate oxygen generation and flow into the delivery device 126. Particularly, oxygen may flow through the oxygen-supply line 124, into the delivery chamber 130, and exit through the array 132 of hollow microneedles. The microneedles may penetrate the stratum corneum such that the flow of oxygen may continue directly into subdermal regions and tissues and, in some embodiments, be absorbed into the bloodstream.

Referring now to FIG. 3, another embodiment of a supply source 102 in accordance with the present invention may include an oxygen reservoir 300 retained within a housing 112. In some embodiments, the oxygen reservoir 300 and/or housing 112 may be commercially available, and may be replaceable or refillable to facilitate a sufficient oxygen supply.

In one embodiment, for example, an oxygen reservoir 300 may retain enough oxygen to sustain a person for ten minutes. In another embodiment, the oxygen reservoir may retain an amount of oxygen sufficient to provide one-third of the required oxygen supply for thirty minutes.

In some embodiments, a flow or pressure regulator 302 may mediate a flow of oxygen from the oxygen reservoir 300 to the delivery device 126. As shown, for example, the flow regulator 302 may be coupled to an end of the oxygen reservoir 300 to regulate a flow of oxygen to an oxygen-supply line 124. The flow regulator 302 may be manually or automatically adjusted according to a desired flow rate. The flow regulator 302 may then permit oxygen to flow at the desired rate into the oxygen-supply line 124 for receipt into the delivery chamber 130 and delivery via the array 132 of microneedles. In other embodiments, the flow regulator 302 may communicate with other delivery devices 126 and/or transport enhancement elements 128.

In any case, in some embodiments, a pressure relief check valve (not shown) may be coupled to the delivery chamber 130 or other delivery device 126 to prevent the pressure from exceeding a safe level at the point of delivery. In other embodiments, a volume of water and a filter may be interposed between the oxygen supply source 102 and the delivery device 126 to humidify the oxygen prior to delivery.

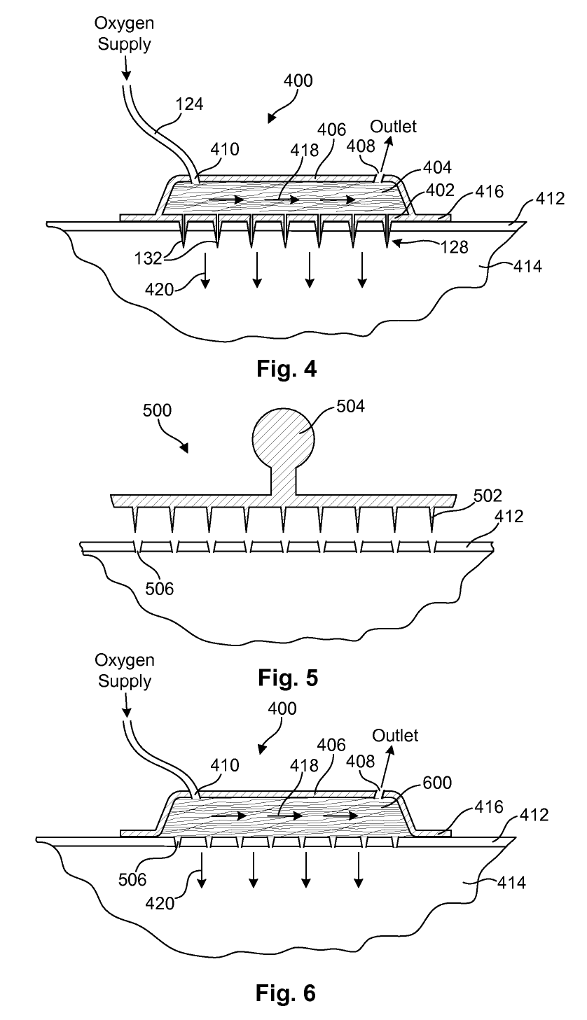

Referring now to FIG. 4, one embodiment of a delivery device 126 may include a patch 400 for topical application. In certain embodiments, the patch 400 may include a contact layer 402, an intermediate layer 404, and a barrier layer 406. The contact layer 402 may directly contact a skin surface 412 and may be substantially porous or perforated to enable oxygen transport therethrough. The contact layer 402 may include, for example, Dermanet®, Mepitel®, Tegapore®, Drynet®, or other suitable material known to those in the art.

The intermediate layer 404 may include a non-woven fabric or woven mesh that may permit oxygen to flow therethrough. In some embodiments, for example, the intermediate layer 404 may include polyester, rayon, nylon, or combinations thereof, or any other suitable material known to those in the art.

The barrier layer 406 may substantially contain oxygen within the patch 400 and prevent outside gases and contaminants from entering the patch 400. In some embodiments, the barrier layer 406 may be substantially impermeable to gases. The barrier layer 406 may be constructed of polyurethane, polyethylene, polypropylene, polyvinyl chloride, Topas® Advanced Polymers, or combinations thereof, for example.

In certain embodiments, a flange 416 or adhesive layer may extend radially outwardly from the barrier layer 406 to substantially seal a perimeter of the patch 400 to the skin surface 412. Like the barrier layer 406, the flange 416 may be substantially gas-impermeable.

In one embodiment, an oxygen-supply line 124 may direct oxygen from a supply source 102 to an inlet 410 or port in the patch 400. The oxygen may proceed through the patch 400 in a substantially horizontal direction 418 towards a gas outlet 408 integrated into the barrier layer 406. This substantially horizontal 418 flow of oxygen may carry with it moisture from the skin or wound enclosed by the patch 400, thereby performing a self-cleaning and detoxifying function.

As shown, a transport enhancement element 128 may include an array 132 of hollow microneedles integrated with or attached to the contact layer 402 of the patch 400. The contact layer 402 is gas-permeable. In one embodiment, the array 132 of hollow microneedles may extend through the contact layer 402 to provide a passageway for oxygen to diffuse from within the patch into subdermal tissues and regions 414.

Particularly, oxygen may proceed through the array 132 in a substantially vertical direction 420, such that the oxygen may be absorbed into localized subdermal regions and tissues 414. In some embodiments, the oxygen may be further absorbed into the bloodstream and distributed throughout the body via the circulatory system.

Referring now to FIG. 5, some embodiments of the present invention may include a transport enhancement element 128 that is independent of the delivery device 126. For example, one embodiment of a transport enhancement element 128 may include a tool 500 equipped to create micro-passageways 506 through the least permeable layer of the skin, the stratum corneum 412.

In some embodiments, the tool 500 may include a handle 504 attached to an array of solid or hollow microneedles 502. A user may grasp the handle 504 to apply the tool 500 to the skin surface 412 such that the microneedles 502 penetrate the stratum corneum 412 to create the passageways 506. The tool 500 may be removed from the skin surface 412 to expose the passageways 506, or may be retained therein.

Indeed, removing the tool 500 after just ten seconds in the skin 412 may leave a perforation pathway that dramatically increases skin permeability. This perforation pathway may enable applied and even ambient oxygen to diffuse towards the fluids in the body exhibiting a lower oxygen partial pressure. At the same time, application of the tool 500 may create a pathway for carbon dioxide to diffuse out, since the partial pressure of carbon dioxide within the body is greater than in the atmosphere.

Referring now to FIG. 6, a patch 400 may be applied to a previously-treated skin surface 412 to increase oxygen permeability and facilitate oxygen transport into subdermal skin layers and tissues 414. In one embodiment, for example, the skin surface 412 may have been previously treated with a tool 500, such as that shown in FIG. 5. As a result, the stratum corneum 412 or skin surface 412 may have passageways 506 integrated therein to increase skin permeability.

The patch 400 may be applied to the skin surface 412 and actuated such that oxygen may be directed from an oxygen supply source 102 into the patch 400 via an oxygen-supply line 124. As previously discussed, the patch 400 may include a barrier layer 406 to both prevent oxygen from getting out of the patch 400, and prevent outside contaminants from getting in. An inlet 410 may be integrated into the barrier layer 406 to permit oxygen from the supply source 102 to enter.

In some embodiments, the patch 400 may further include a porous layer 600 that substantially integrates the contact 402 and intermediate layers 404 of previously-discussed embodiments. For example, the porous layer 600 may be substantially compatible with the skin surface 412 to avoid sticking, while facilitating oxygen diffusion through the patch 400 and into subdermal regions and tissues 414. In some embodiments, the porous layer 600 may include, for example, Dermanet®, Mepitel®, Tegapore®, Drynet®, polyester, rayon, nylon, combinations thereof, and the like. The porous layer 600 may enable the oxygen to diffuse into the previously-created passageways 506. The oxygen may then be absorbed in a substantially vertical direction 420 into subdermal regions and tissues 414.

As previously discussed, the oxygen may also proceed in a substantially horizontal direction 418 from the inlet 410 to an outlet 408 integrated into the barrier layer 406. This flow of oxygen may accumulate and remove excess water particles and other debris in transit.

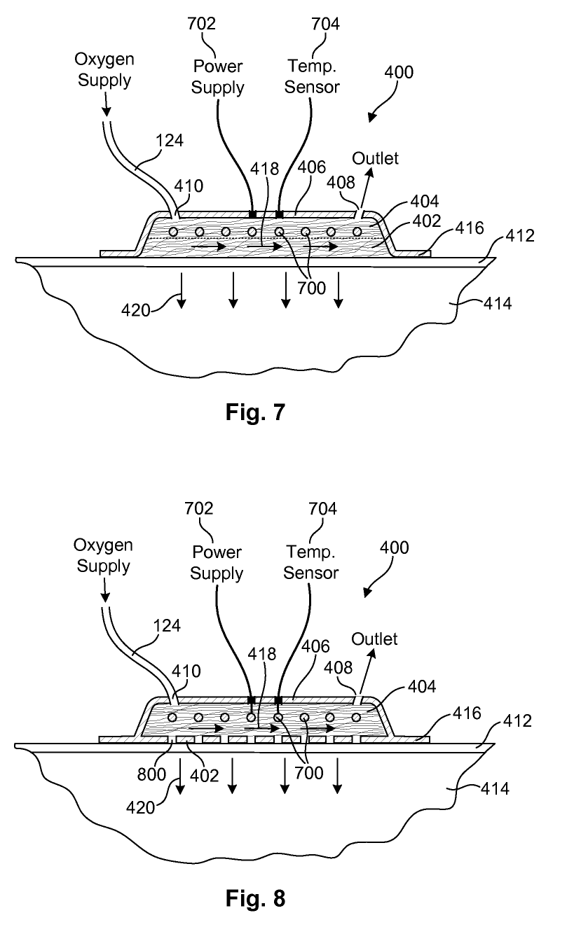

Referring now to FIG. 7, another embodiment of a transport enhancement element 128 in accordance with the invention may include a heat-generating device 700 integrated into or coupled to the delivery device 126 or patch 400 to increase skin permeability. The heat-generating device 700 may apply direct or indirect heat to a localized area of skin identified for transdermal oxygen delivery. In one embodiment, the heat-generating device is configured to raise the temperature of the localized area to between about 41 degrees Celsius and about 43 degrees Celsius.

In some embodiments, for example, the heat-generating device 700 may include one or more electrical resistive wires or heating elements adapted to heat the patch 400 to a predetermined temperature. The electrical resistive wires may be connected to a power supply 702 that, in some embodiments, may be coupled to a temperature control system (not shown) to monitor and control the temperature of the patch 400.

For example, in one embodiment, the temperature control system may include a temperature sensor 704, such as a thermocouple, situated within the patch 400 to sense the temperature of the patch 400. In certain embodiments, the temperature sensor 704 may be situated proximate to the area of skin 412 being treated such that the temperature sensed substantially reflects the temperature of the skin 412. Using this temperature reading, the temperature control system may communicate with the power supply 702 to adjust the power supplied to the heat-generating device 700 in response to the temperature detected by the temperature sensor 704. For example, the temperature control system may adjust the voltage supplied to the heat-generating device 700 or adjust the duty cycle of the voltage supplied to the heat-generating device 700 to adjust the temperature.

Referring now to FIG. 8, an alternative embodiment of a delivery device 126 or patch 400 may include a contact layer 402 having perforations 800 or channels therein to facilitate transdermal oxygen transport. In some embodiments, the perforated contact layer 402 and barrier layer 406 may be substantially monolithic in nature, such that the perforated contact layer 402 and the barrier layer 406 comprise the same substantially gas-impervious material. Alternatively, the perforated contact layer 402 may include a porous or breathable material, or any other suitable material known to those in the art.

The perforations 800 integrated into the contact layer 402 may channel oxygen retained within the patch 400 towards a localized area of skin beneath the patch 400. A flange 416 may extend outwardly from the barrier layer 406 to substantially seal a perimeter of the patch 400 around the localized area.

As in certain other embodiments, the patch 400 may receive a supply of oxygen from a supply source 102. The oxygen may be received by an inlet 410 in the barrier layer 406, and may diffuse through an intermediate layer 404. The perforations 800 in the contact layer 402 may then enable oxygen to be absorbed into localized subdermal regions and tissues 414. Oxygen may also vent through an outlet 408 in the barrier layer 406.

In some embodiments, the patch 400 may include a transport enhancement element 128 to further facilitate transdermal oxygen delivery. The transport enhancement element 128 may include, for example, a heat-generating device 700 having electrical resistive wires powered by a power supply 702 and controlled by a temperature control system communicating with a temperature sensor 704 to maintain a predetermined temperature within the patch 400. In other embodiments, the transport enhancement element 128 may include a topical substance applied to the localized area of skin to increase skin permeability. The topical substance may include nitroglycerin, skin permeation enhancers, such as dimethyl sulphoxide (DMSO), and 1-[2-(decylthio)ethyl]azacyclopentan-2-one (HPE-101), or topical substances sold under the trademarks Labrafac CC, Labrafil, Labrasol and Transcutol that are known to enhance skin permeability. In one embodiment, a concentration of 10% (wt./wt.) of the preceding skin enhancers may be used as part of the topical substance.

In still other embodiments, as discussed in more detail above, the transport enhancement element 128 may include an array of microneedles or other mechanical device applied to the localized area of skin 412 to increase skin permeability.

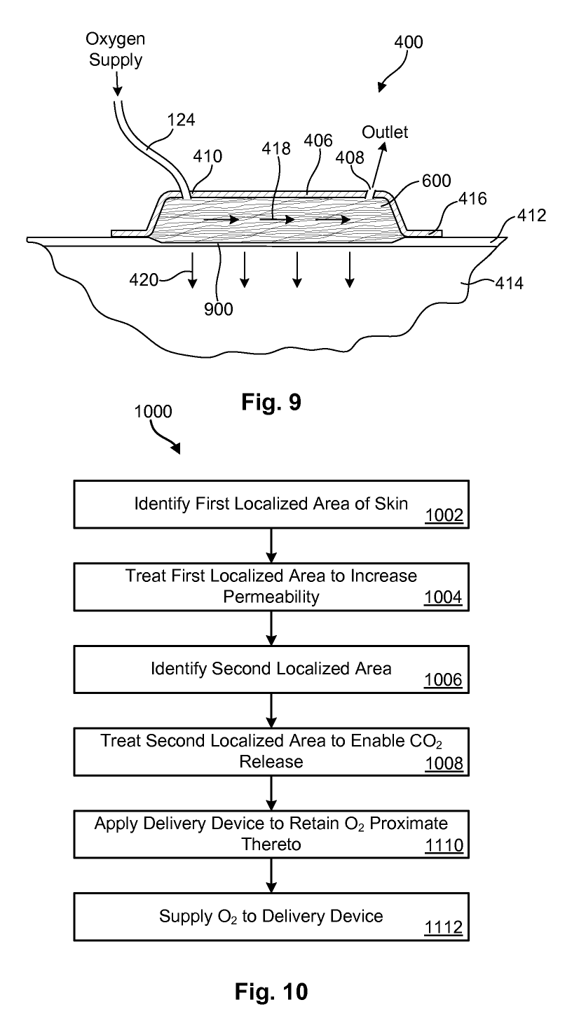

In one embodiment, the transport enhancement element 128 may comprise a skin reduction device applied to the localized area prior to transdermal oxygen transport. Referring now to FIG. 9, application of such a skin reduction device (not shown) may reduce a thickness 900 of the stratum corneum 412 to facilitate oxygen transport into subdermal regions and tissues 414. Specifically, reduction of the stratum corneum 412 may reduce biological resistance to transdermal oxygen transport.

In certain embodiments, as shown, a porous layer 600 may further facilitate oxygen diffusion into subdermal tissues 414. In other embodiments, a perforated or porous contact layer 402 may directly contact the reduced thickness 900 of the stratum corneum 412. The contact layer 402 may include a material that readily permits oxygen transport therethrough, while minimizing interference with the reduced skin surface 412. For example, the contact layer 402 may include Dermanet®, Mepitel®, Tegapore®, Drynet®, or any other suitable material known to those in the art.

Referring now to FIG. 10, a method 1000 for facilitating transdermal oxygen delivery in accordance with certain embodiments of the invention may be used to promote wound healing and/or sustain life. In some embodiments, the method 1000 may include identifying 1002 a first localized area of skin and treating 1004 the area to increase its oxygen permeability. Treating 1004 the area may include, for example, applying an array of microneedles or other mechanical device to the localized area to create passageways for oxygen to be transported to subdermal regions and tissues, applying a topical substance of the kind discussed above or heat to increase skin permeability, or reducing a dermal thickness of the localized area. The heat may be applied to raise the temperature of the localized area to between about 41 degrees Celsius and about 43 degrees Celsius.

In certain embodiments, the method 1000 may further include identifying 1006 a second localized area of skin and treating 1008 the second localized area to enable release of carbon dioxide from the body. Treating 1008 the second localized area may include, for example, applying an array of microneedles or other mechanical device to the skin surface to create passageways for carbon dioxide to diffuse across its concentration gradient from within the body to the outside environment.

In some embodiments, the first and second localized areas of skin may be substantially the same. In other embodiments, the array of microneedles may be iteratively applied to various localized areas to further facilitate carbon dioxide release from the body and thereby facilitate a life-sustaining function.

A delivery device may be applied 1110 over the first localized area to retain oxygen proximate thereto. For example, the delivery device may include a patch substantially sealed over a perimeter of the first localized area. The patch may include a barrier layer to retain oxygen within the patch and bar entry to outside gases and contaminants. Finally, oxygen may be supplied 1112 to the delivery device for delivery to the first localized area.

The following are several non-limiting examples of methods contemplated for facilitating transdermal oxygen delivery in accordance with the invention:

### EXAMPLE 1

Respiration may be impaired due to a damaged or collapsed trachea. To temporarily provide oxygen and promote removal of carbon dioxide from the patient as needed to sustain life, a patch having a hollow microneedle array, with microneedles spaced approximately 0.5 cm apart, may be applied to an area of intact skin in accordance with embodiments of the invention. Optionally, the temperature of the patch may be controlled to 42° C.±1° C. Warm sterile oxygen may be supplied to the area at a rate of about two hundred fifty cubic centimeters per minute from a battery-operated supply source utilizing a solid oxide electrolyte membrane. The oxygen may flow through the patch and vent at the opposite end thereof, carrying away excess moisture in transit. Additionally, an array of solid microneedles may be used to perforate the skin or mucosa in several areas. The array of solid microneedles may be withdrawn from the skin or mucosa to permit carbon dioxide release. To further enhance oxygen delivery, the patient may also breathe 100% oxygen, supplied through a mask and generated by the same supply source.

### EXAMPLE 2

A wound surface may be cleaned and not exudating. To stimulate healing and to prevent infection, the wound and about six inches of skin around the wound perimeter may be enclosed in a patch in accordance with certain embodiments of the invention. The temperature of the patch may be controlled to 42° C.±1° C. Warm sterile oxygen may be supplied to the wound at a rate of about ten cubic centimeters per minute from a battery-operated supply source utilizing a solid oxide electrolyte membrane. The oxygen may flow through the patch and vent at the opposite end thereof, carrying away excess moisture in transit. Additionally, the patient may breathe 100% oxygen, supplied through a mask and generated by the same supply source. Such oxygen may be supplied at the rate of approximately three liters per minute for one hour, three times per day. The wound may be inspected two times per week to measure the progress until healed.

### EXAMPLE 3

A wound surface may be cleaned and not exudating. To stimulate healing and to prevent infection, the wound and about six inches of skin around the wound perimeter may be enclosed by a patch. Hollow microneedles spaced approximately 0.5 cm apart may be applied to intact skin surrounding the wound. Warm sterile oxygen may be supplied to the wound at a rate of approximately ten cubic centimeters per minute from a battery-operated supply source utilizing a solid oxide electrolyte membrane. The oxygen may flow through the bandage and vent at the opposite end thereof, carrying away excess moisture in transit. Additionally, the patient may breathe 100% oxygen, supplied through a mask and generated by the same supply source. Such oxygen may be supplied at the rate of approximately three liters per minute for one hour, three times per day. The wound may be inspected two times per week to measure the progress until healed.

### EXAMPLE 4

A wound surface may be cleaned and not exudating. To stimulate healing and to prevent infection, the wound and about six inches of skin around the wound perimeter may be enclosed by a patch. The intact skin surrounding the wound may be prepared by skiving approximately ten microns of thickness from the stratum corneum, using a device commonly used to remove skin for skin grafts. Warm sterile oxygen may be supplied to the wound at a rate of approximately ten cubic centimeters per minute from a battery-operated supply source utilizing a solid oxide electrolyte membrane. The oxygen may flow through the patch and vent at the opposite end thereof, carrying away excess moisture in transit. Additionally, the patient may breathe 100% oxygen, supplied through a mask and generated by the same supply source. Such oxygen may be supplied at the rate of approximately three liters per minute for one hour, three times per day. The wound may be inspected two times per week to measure the progress until healed.

### EXAMPLE 5

A wound surface may be cleaned and not exudating. To stimulate healing and to prevent infection, the wound and about six inches of skin around the wound perimeter may be enclosed in a patch. The temperature of the patch may be controlled to 42° C.±1° C. Prior to applying the patch, the intact skin may be prepared by applying dimethyl sulfoxide to the surface to increase skin permeability. Warm sterile oxygen may be supplied to the wound at a rate of approximately ten cubic centimeters per minute from a battery-operated supply source utilizing a solid oxide electrolyte membrane. The oxygen may flow through the patch and vent at the opposite end thereof, carrying away excess moisture in transit. Additionally, the patient may breathe 100% oxygen, supplied through a mask and generated by the same supply source. Such oxygen may be supplied at the rate of approximately three liters per minute for one hour, three times per day. The wound may be inspected two times per week to measure the progress until healed.

### EXAMPLE 6

An individual with Type 1 diabetes may be implanted with a microporous bag containing porcine pancreatic islet cells. The bag may be implanted subcutaneously. To ensure the islet cells receive sufficient oxygen for survival, the skin above the implant and about six inches of skin around the implant perimeter may be enclosed by a patch. The temperature may be controlled to 42° C.±1° C. Warm sterile oxygen may be supplied to the wound at a rate of approximately ten cubic centimeters per minute from a battery-operated supply source utilizing a solid oxide electrolyte membrane. The oxygen may flow through the patch and vent at the opposite end thereof, carrying away excess moisture in transit. Additionally, the patient may breathe 100% oxygen, supplied through a mask and generated by the same supply source. Such oxygen may be supplied at the rate of approximately three liters per minute for fifteen minutes, six times per day. Glucose levels may be monitored periodically to ensure the survival of the islet cells and control of diabetes.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus for facilitating transdermal oxygen delivery, the apparatus comprising:
a supply source to provide a supply of oxygen;
a delivery device coupled to the supply source to deliver the oxygen transdermally through the skin of a patient, wherein the delivery device comprises:
a barrier layer to substantially retain the oxygen over a localized area of the skin; and
a gas-permeable contact layer to deliver the oxygen to the localized area; and
a transport enhancement element to increase the oxygen permeability of the localized area.

2. The apparatus of claim 1, wherein the supply source comprises at least one of an oxygen generator and an oxygen reservoir.

3. The apparatus of claim 2, wherein the oxygen generator produces oxygen by at least one of a chemical reaction and an electrical-current-induced reaction.

4. The apparatus of claim 1, wherein the barrier layer is substantially impermeable to a flow of gases thereacross.

5. The apparatus of claim 1, wherein the gas-permeable contact layer comprises an array of substantially hollow microneedles.

6. The apparatus of claim 5, wherein at least one of the microneedles comprises a length ranging between about ten microns and about one thousand microns.

7. The apparatus of claim 5, wherein at least one of the microneedles comprises a cross-sectional dimension ranging between about ten microns and about one hundred microns.

8. The apparatus of claim 5, wherein at least one of the microneedles comprises an inner diameter ranging between about three microns and about eighty microns.

9. The apparatus of claim 1, further comprising a control device to control a rate at which the oxygen is delivered to the localized area.

10. The apparatus of claim 1, wherein the transport enhancement element comprises an array of microneedles to selectively perforate the localized area.

11. The apparatus of claim 10, wherein at least one of the microneedles comprises a length ranging between about ten microns and about one thousand microns.

12. The apparatus of claim 10, wherein at least one of the microneedles comprises a cross-sectional dimension ranging between about ten microns and about one hundred microns.

13. The apparatus of claim 10, wherein at least one of the microneedles comprises an inner diameter ranging between about three microns and about eighty microns.

14. The apparatus of claim 1, wherein the transport enhancement element comprises a heat-generating device to apply heat to the localized area.

15. The apparatus of claims 1, wherein the heat-generating device is configured to raise the temperature of the localized area to between about 41 degrees Celsius and about 43 degrees Celsius.

16. The apparatus of claim 1, wherein the transport enhancement element comprises a reduction device to selectively reduce a skin thickness of the localized area.

17. The apparatus of claim 1, wherein the transport enhancement element comprises a topical substance to increase permeability of the localized area.

18. The apparatus of claim 17, wherein the topical substance comprises at least one of nitroglycerin, dimethyl sulphoxide, 1-[2-(decylthio)ethyl]azacyclopentan-2-1, and combinations thereof.

19. A method for facilitating transdermal oxygen delivery, the method comprising:
identifying a localized area of skin;
treating the localized area to increase its oxygen permeability;
applying a delivery device over the localized area to substantially retain oxygen proximate thereto; and
supplying oxygen to the delivery device for delivery to the localized area.

20. The method of claim 19, wherein supplying the oxygen comprises generating the oxygen by at least one of a chemical reaction and an electrical-current-induced reaction.

21. The method of claim 19, wherein supplying the oxygen comprises accessing an oxygen reservoir.

22. The method of claim 19, wherein treating the localized area comprises perforating the localized area with an array of microneedles.

23. The method of claim 22, wherein at least one of the microneedles comprises a length ranging between about ten microns and about one thousand microns.

24. The method of claim 22, wherein at least one of the microneedles comprises a cross-sectional dimension ranging between about ten microns and about one hundred microns.

25. The method of claim 22, wherein at least one of the microneedles comprises an inner diameter ranging between about three microns and about eighty microns.

26. The method of claim 19, wherein treating the localized area comprises applying heat to the localized area.

27. The method of claim 26, wherein applying heat to the localized area comprises heating the localized area to between about 41 degrees Celsius to about 43 degrees Celsius.

28. The method of claim 19, wherein treating the localized area comprises reducing a skin thickness.

29. The method of claim 19, wherein treating the localized area comprises applying a topical substance to the localized area to increase its permeability.

30. The method of claim 29, wherein the topical substance comprises at least one of nitroglycerin, dimethyl sulphoxide, 1-[2-(decylthio)ethyl]azacyclopentan-2-1, and combinations thereof.

31. A method for facilitating transdermal oxygen delivery, the method comprising:
identifying a first localized area of skin;
treating the first localized area to increase its oxygen permeability;
identifying a second localized area of skin;
treating the second localized area to enable release of carbon dioxide;
applying a delivery device over the first localized area to substantially retain oxygen proximate thereto; and
supplying oxygen to the delivery device for delivery to the first localized area.

32. The method of claim 31, wherein treating the first localized area comprises at least one of perforating, applying heat, reducing a skin thickness, and applying a topical substance to the first localized area to increase its oxygen permeability.

33. The method of claim 31, wherein treating the second localized area comprises at least one of perforating, applying heat, reducing a skin thickness, and applying a topical substance to the second localized area to enable release of carbon dioxide.
